(19) United States
(12) Patent Application Publication
(10) Pub. No.: US 2019/0201848 A1
(43) Pub. Date: Jul. 4, 2019

(54) DEVICE FOR BLOOD COLLECTION

(71) Applicant: HunchDX, LLC, Dallas, TX (US)

(72) Inventor: Donald Rao, Dallas, TX (US)

(21) Appl. No.: 16/238,283

(22) Filed: Jan. 2, 2019

Related U.S. Application Data
(60) Provisional application No. 62/612,985, filed on Jan. 2, 2018.

(51) Int. Cl. B01D 63/02; A61B 5/15

(52) U.S. Cl. CPC B01D 63/025; A61B 5/150755; A61B 5/150534; B01D 2313/243; A61B 5/150251; A61B 5/150236; B01D 2315/10; A61B 5/150099

(57) ABSTRACT

The present invention includes a filter apparatus including a filtration unit; and a collection chamber in fluid communication with the filtration unit; wherein a vacuum draws the biological fluid through the apparatus; and wherein selected biological material comprised by the biological fluid is separated from a remainder of the biological fluid.

### CROSS-REFERENCE TO RELATED APPLICATIONS

This application is a non-provisional patent application of and claims priority to U.S. provisional patent application Ser. No. 62/612,985 filed on Jan. 2, 2018 and entitled “Device for Blood Collection”, the contents of which are hereby incorporated by reference in their entirety.

### STATEMENT OF FEDERALLY FUNDED RESEARCH

None.

### TECHNICAL FIELD OF THE INVENTION

The present invention relates in general to the field of the separation of biological fluids, and more particularly, to devices and methods for the separation of cells and parts thereof, bacteria, viruses and the like from a biological fluid.

### BACKGROUND OF THE INVENTION

Without limiting the scope of the invention, its background is described in connection with blood collection devices.

Liquid biopsy for diagnostics is a non-invasive method for detecting cancer, infectious agents or genetic disorders. Recent advancement in DNA sequencing technology greatly enhanced the sensitivity of detection, and hence the strong interests in DNA based diagnostic with liquid biopsies. Cell-free DNA (cfDNA) in miniscule amount can be detected and identified (1, 2, 3). For cfDNA analysis, the blood collection and blood sample processing is extremely important to maintain the integrity of samples for accurate analysis. More than 10 ml of blood is often required for an accurate analysis.

Current blood collection and sample processing are based on decade old technologies; it involves many steps and requires manual operations for which leads to variations in recovery and quality of recovered materials. Although few developments were designed to improve the quality of recovered materials by adding stabilizing agents, it is still cumbersome with variable results.

For many diagnostics analysis, it requires obtaining either plasma or serum clear from cells in the blood. The current conventional process is to collect blood into a vacuum blood collection tube (e.g., VACUTAINER®) contain anti-clotting agents with subsequent centrifugation process to separate serum/plasma from cells and then manually withdraw serum layer without disturbing buffy coat and cell layer. Mechanistically, the blood sample fractionation process is manual-driven which requires trained personnel to handle blood samples and further process in clinical labs equipped with centrifuge. The timing and handling of sample are critical for sample integrity and recovery efficiency. Blood cells can rupture during the sample processing steps to release whole cell DNA/RNA or heme and other cellular components interfere with downstream assays (4, 7). New improvements are to add preservative agents to maintain the integrity of sample for a longer period of time before manual processing; however, those improvements still require downstream manual-driven processing. With manual-driven, the result varies from operator to operator. The centrifugation has to be carefully done with appropriate g-force and time in order to pellet blood cells without rupture. Serum/plasma fraction needs to be withdrawn with care to not disturb the pelleted cells and the buffet coat layer.

Thus, a need remains for simple blood collection and separation devices that can be used without extensive equipment (e.g., centrifugation), refrigeration, etc.

### SUMMARY OF THE INVENTION

This invention describes a novel device for effective separation of serum/plasma from blood cells while blood is being collected or from a blood collection tube soon after blood collection. The device is one-step process accomplished at bedside without any clinical instrument.

In one embodiment, the present invention includes an apparatus comprising: a wetted tangential flow filtration unit capable of separating the cells from a biological fluid by tangential flow filtration; and a vacuum-assisted collection chamber connected to the tangential flow filtration unit, wherein a vacuum draws the biological fluid from the source through the tangential flow filtration unit to separate the cells from the biological fluid. In another aspect, the present invention consists essentially of a wetted tangential flow filtration unit capable of separating the cells from a biological fluid by tangential flow filtration; and a vacuum-assisted collection chamber connected to the tangential flow filtration unit, wherein a vacuum draws the biological fluid from the source through the tangential flow filtration unit to separate the cells from the biological fluid. In one aspect, the vacuum-assisted collection chamber collects the cells, and the wetted tangential filtration unit collects a cell-free fluid. In another aspect, the vacuum-assisted collection chamber collects a cell-free fluid and the wetted tangential filtration unit collects cells. In another aspect, a source of the biological fluid further comprises a collection chamber upstream from the wetted tangential flow filtration unit, wherein the wetted tangential flow filtration unit further comprises a first and a second port downstream from the wetted tangential flow filtration unit to which a first and a second vacuum collection chamber is capable of connecting, wherein the source of a biological fluid in the upstream collection chamber comprises a needle to facilitate communication with the tangential flow filtration unit. In another aspect, the source of the biological fluid comprises a needle surrounded by a shroud. In another aspect, the wetted tangential flow filtration unit further comprises a first port that is in fluid communication with a sample flow-through and the second port is in fluid communication with a sample permeate from the wetted tangential flow filtration unit. In another aspect, the collection chamber further comprises one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, protease/s or salts. In another aspect, the first and second ports are, alternatively, a female or a male connector. In another aspect, the first and second ports operationally connect via leak-free connections between a male-taper fitting and its mating female, or vice versa. In another aspect, the collection of cells or a cell-free fluid does not require electrical power. In another aspect, the vacuum-assisted collection chamber is a syringe. In another aspect, the vacuum-assisted collection chamber is a single syringe with two separate concentric chambers and plungers. In another aspect, the vacuum-assisted collection chamber is a vacuum blood collection tube that optionally comprises one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, protease/s or salts. In another aspect, the apparatus is adapted to connect to a needle within a blood collection safety shroud. In another aspect, the wetted tangential flow filtration unit comprises a tangential flow filtration membrane, cassette, or hollow-fiber. In another aspect, the wetted tangential flow filtration unit is sized to separate a target cell, cellular organelle, bacteria, or virus from the biological fluid. In another aspect, a source of blood is a vacuum blood collection tube. In another aspect, the biological fluid is selected from whole blood, sputum, cerebrospinal fluid, tears, urine, feces, or biopsy. In another aspect, the apparatus is disposable.

In another embodiment, the present invention includes a method of separating cells from a biological fluid comprising: connecting a wetted tangential flow filtration unit to the source of biological fluid comprising cells and capable of separating the cells from the biological fluid by tangential flow filtration; connecting a vacuum-assisted collection chamber to the tangential flow filtration unit, wherein a vacuum draws the biological fluid from the source through the tangential flow filtration unit to separate the cells from the biological fluid; and separating the cells from the biological fluid by tangential flow filtration into a retentate comprising the cells and a permeate that comprises a cell-free fluid. In one aspect, the vacuum-assisted collection chamber collects the cells, and the wetted tangential filtration unit collects a cell-free fluid. In another aspect, the vacuum-assisted collection chamber collects a cell-free fluid, and the wetted tangential filtration unit collects cells. In another aspect, a source of the biological fluid further comprises a collection chamber upstream from the wetted tangential flow filtration unit, wherein the wetted tangential flow filtration unit further comprises a first and a second port downstream from the wetted tangential flow filtration unit to which a first and a second vacuum collection chamber is capable of connecting, wherein the source of blood is a patient and the blood collection chamber comprises a needle in fluid communication with the collection chamber. In another aspect, the source of the biological fluid operationally connects to the wetted tangential flow filtration unit that comprises a needle surrounded by a shroud. In another aspect, the wetted tangential flow filtration unit further comprises a first port that is in fluid communication with a source of retained cells and the second port is in fluid communication with a sample permeate from the wetted tangential flow filtration unit. In another aspect, the collection chamber further comprises one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, protease/s or salts. In another aspect, the first and second ports are, alternatively, a female or a male connector. In another aspect, the first and second ports operationally connect via leak-free connections between a male-taper fitting and its mating female, or vice versa. In another aspect, the collection of cells or a cell-free fluid does not require electrical power. In another aspect, the vacuum-assisted collection chamber is a syringe. In another aspect, the vacuum-assisted collection chamber is a single syringe with two separate concentric chambers and plungers. In another aspect, the vacuum-assisted collection chamber is a vacuum blood collection tube that optionally comprises one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, protease/s or salts. In another aspect, the apparatus is operationally connected to a needle within a blood collection safety shroud. In another aspect, the wetted tangential flow filtration unit comprises a tangential flow filtration membrane, cassette, or hollow-fiber. In another aspect, the wetted tangential flow filtration unit is sized to separate a target cell, cellular organelle, bacteria, or virus from the biological fluid. In another aspect, the source of biological fluid is a vacuum blood collection tube. In another aspect, the biological fluid is selected from whole blood, sputum, cerebrospinal fluid, tears, urine, feces, or biopsy.

In another embodiment, the present invention includes a kit comprising: a wetted tangential flow filtration unit capable of separating the cells from a biological fluid by tangential flow filtration; and a vacuum-assisted collection chamber connected to the tangential flow filtration unit, wherein a vacuum draws the biological fluid from the source through the tangential flow filtration unit to separate the cells from the biological fluid.

In another embodiment, the present invention includes a filter apparatus including a filtration unit; and a collection chamber in fluid communication with the filtration unit; wherein a vacuum draws the biological fluid through the apparatus; and wherein selected biological material included in the biological fluid is separated from a remainder of the biological fluid. In one aspect, the selected biological material is substantially retained by the filtration unit and the remainder of the biological fluid is substantially passed to the collection chamber or the selected biological material is passed to the collection chamber and the remainder of the biological fluid is substantially retained by the filtration unit. In another aspect, the filtration unit includes a hollow-fiber filtration unit, a membrane filtration unit, or a cassette filtration unit. In another aspect, the filtration unit is wetted prior to packaging. In another aspect, the filtration unit includes a one or more hollow fibers. In another aspect, the one or more hollow fibers include a mixed cellulose ester, polysulfone, plastic polymers or some combination. In another aspect, the one or more hollow fibers are open at the upper ends and closed at the lower ends or closed at the upper ends and open at the lower ends. In another aspect, the filtration unit is disposed with a first tube segment and the collection chamber is disposed within a second tube segment and the first tube segment and the second tube segment are detachably connected to each other; or the filtration unit and the collection chamber are disposed within a single tube. In another aspect, a source of the biological fluid includes a source chamber upstream from the filtration unit and a needle to facilitate fluid communication with the filtration unit. In another aspect, the filter apparatus further includes a first vacuum port and a second vacuum port downstream from the filtration unit, wherein the first vacuum port and the second vacuum port are capable of coupling to a first vacuum collection chamber and a second vacuum collection chamber, respectively. In another aspect, the source of the biological fluid further includes a needle surrounded by a shroud. In another aspect, the filter apparatus further includes a first sample flow-through port in fluid communication with the filtration unit with a sample flow-through and a second sample flow-through in fluid communication with a sample permeate from the filtration unit. In another aspect, the first downstream port and the second downstream port are complementary male and female connectors. In another aspect, the first downstream port and the second downstream port are connected via a male taper-fitting and its mating female. In another aspect, the filtration unit, the collection chamber, or both, further include one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, proteases or salts. In another aspect, the interior surfaces of the filtration unit are positively charged or be negatively charged or the interior surfaces of the collection chamber are positively charged or negatively charged. In another aspect, the apparatus does not require electrical power. In another aspect, the collection chamber is a syringe. In another aspect, the syringe includes two separate concentric chambers and plungers. In another aspect, the apparatus is adapted to couple in fluid connection to a needle within a shroud. In another aspect, the biological fluid includes whole blood, sputum, cerebrospinal fluid, tears, urine, feces, or a biopsy sample. In another aspect, the selected biological material includes cells, cellular organelles, bacteria, viruses, or cell-free DNA. In another aspect, a source of the biological fluid is a vacuum blood collection tube. In another aspect, the apparatus is configured to filter a sample of biological fluid with a volume of 4-10 ml. In another aspect, the apparatus is disposable.

In another embodiment, the present invention includes a filter kit including a filter apparatus including a filtration unit; and a collection chamber in fluid communication with the filtration unit; wherein a vacuum draws the biological fluid through the apparatus; and wherein selected biological material included in the biological fluid is separated from a remainder of the biological fluid.

In another embodiment, the present invention includes a method of filtering a biological fluid including connecting a filtration unit to a source of biological fluid including selected biological material; connecting a collection chamber to the filtration unit; drawing the biological fluid through the filtration unit with a vacuum; and separating the selected biological material from a remainder of the biological fluid by filtration through the filtration unit. In one aspect, the selected biological material is substantially retained by the filtration unit and the remainder of the biological fluid is substantially passed to the collection chamber or the selected biological material is passed to the collection chamber and the remainder of the biological fluid is substantially retained by the filtration unit. In another aspect, the filtration unit includes a hollow-fiber filtration unit, a membrane filtration unit, or a cassette filtration unit. In another aspect, the filtration unit includes one or more hollow fibers. In another aspect, the one or more hollow fibers include a mixed cellulose ester, polysulfone, plastic polymers or some combination.

### BRIEF DESCRIPTION OF THE DRAWINGS

For a more complete understanding of the features and advantages of the present invention, reference is now made to the detailed description of the invention along with the accompanying figures and in which:

FIG. 1A shows a prior art method of obtaining a blood sample, separating plasma from cells, and transferring the plasma to a tube.

FIG. 1B shows a method of obtaining a blood sample and separating plasma from cells such that the plasma is already in a container, according to the present invention.

FIG. 2 shows one example of the blood separation device of the present invention.

FIG. 3 shows a plan view of a plasma/serum and blood cell separation device using the tangential flow filtration (TFF) device of the present invention.

FIG. 4 shows a close-up view of the TFF membrane, in which blood containing blood cells and plasma enter the TFF membrane of the present invention.

FIGS. 5A to 5C show another embodiment, the TFF membrane unit connects to a blood collection apparatus of the present invention. FIG. 5A shows a view of the complete unit from the needle for the blood draw to the final tube and separation device. FIG. 5B shows the TFF membrane after blood cells and plasma has been separated, with either the plasma or blood cells remaining and accessible from the interstitial space surrounding the TFF membrane or inside lumen of the TFF membrane. FIG. 5C shows the collection tube where the plasma separated from the blood cells or blood cells separated from the plasma.

FIGS. 6A and 6B show a filter apparatus in two segments in a single segment and in a single segment.

FIG. 7 shows a flowchart of a method embodiment of the invention.

FIG. 8 depicts two modes of tangential flow filtration.

FIGS. 9A and 9B show views of a tangential flow filtration cartridge.

FIG. 10A and 10B depict the GAPDH protein concentration inside-out test of Example 1 and the hemoglobin concentration for the inside-out test of Example 1, for each experimental fraction, respectively.

FIGS. 11A and 11B depict the GAPDH protein concentration for the outside-in test of Example 1 and the hemoglobin concentration for the outside-in test of Example 1 for each experimental fraction, respectively.

FIG. 12 shows two small filtration units.

FIG. 13 depicts a result using a small filtration unit and centrifugation.

FIG. 14 shows filtration units in conventional BD blood collection tubes.

FIG. 15 illustrates an electropherogram of comparative cell-free DNA yields using a conventional method and a method of the present invention.

FIG. 16 depicts a test filtration apparatus.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

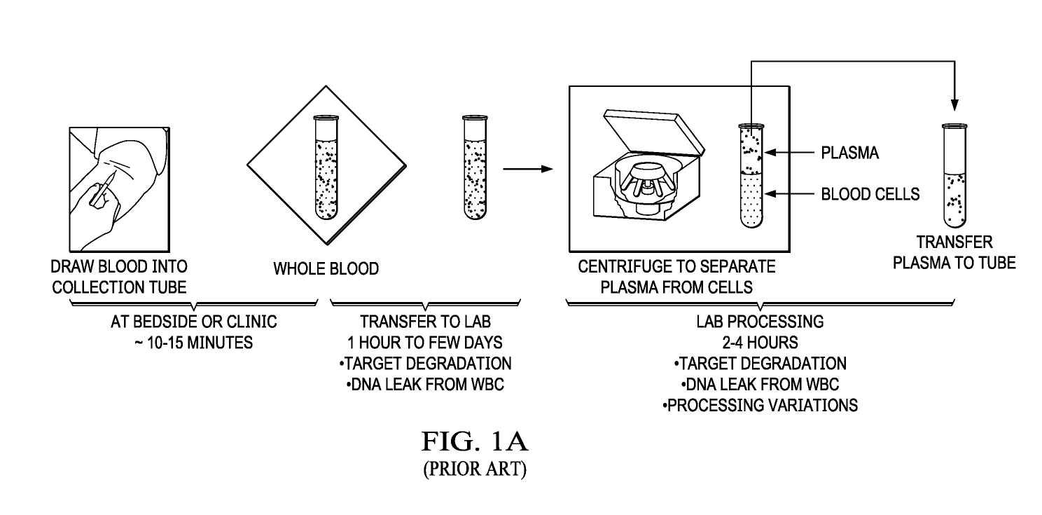

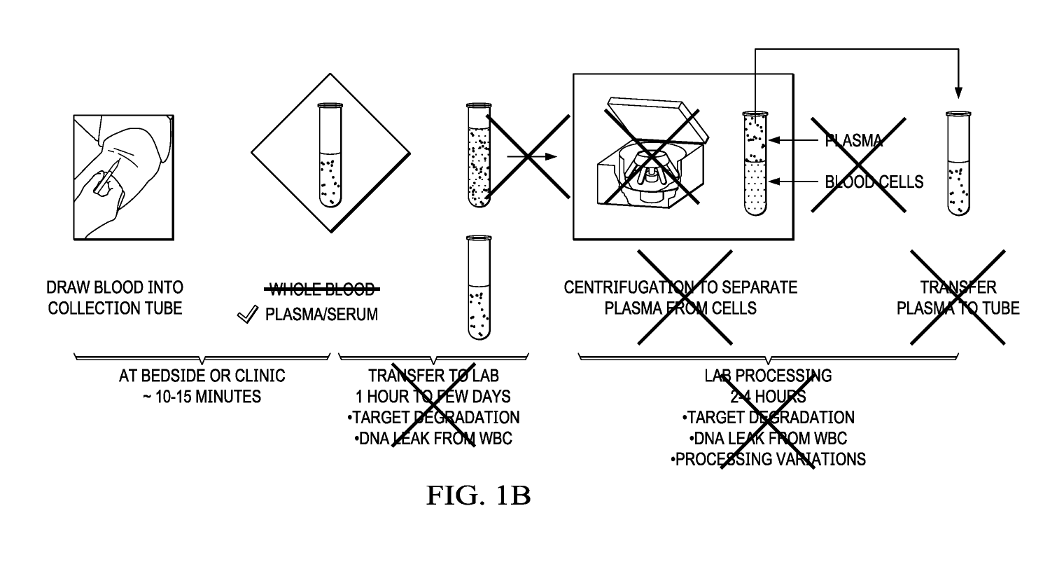

Prior art methods to obtain plasma from blood typically require a number of steps and time to perform them. One such method is shown in FIG. 1A. Typically, whole blood is drawn from a patient into a collection tube, which generally takes about 10-15 minutes at the patient's location. The collection tube is then generally sent to a lab to be placed in a centrifuge to separate the blood cells from the plasma. Sending the blood to a lab typically takes one hour to a few days, during which the sample degrades and DNA can leak from the whole blood cells. Centrifuging the sample often takes about 2-4 hours, during which time the sample degrades and is subject to processing variations, and DNA can leak from the whole blood cells. The plasma is manually transferred to another tube where operational efficiency differs from operator to operator.

The present invention eliminates all of these steps but the initial sample collection, adding only a step of filtering the sample at the place and time of collection to separate blood cells from plasma, as shown in FIG. 1B. Sample collection and filtering with the present invention generally takes about 10-15 minutes at the location of the patient. Collected samples are stable, free from biological material leakage from blood cells and provide standardized plasma collection process.

The present invention can be used in a number of contexts, to filter biological fluid, e.g., whole blood, sputum, cerebrospinal fluid, tears, urine, feces, or a biopsy sample, in order to obtain or to filter out organisms or selected biological materials, where such selected biological materials may include, e.g., cells, cellular organelles, bacteria, viruses, or cell-free DNA.

One particularly promising use is the filtering of cell-free DNA to detect various conditions, e.g., cancer, infectious agents, cardiovascular events, trisomy, fetus genetic defects and the sex of a fetus.

Membrane for blood fractionation is in use for many different purposes such as hemodialysis, plasmaphoresis, plasma exchange, or toxin removal. The cross-flow filtration process passes fluids parallel rather than perpendicular to the filtration membrane to prevent clogging has also been well described in patents. U.S. Pat. No. 4,212,742 proposed the use of unique filtration process for plasmapheresis to separate blood cells from liquid suspension of blood cells by flow along the surface of membrane. U.S. Pat. No. 4,888,115 was for cross-flow filtration process applicable for biological materials such as plasmaphoresis (5). Solomon et al. (6) described the separation of plasma from whole blood by cross flow microfiltration instead of centrifugation. The membrane pore diameters were proposed to be between 0.2 and 0.8 μm in order to permeate the largest immunoglobulins while retaining platelets and red cells. This technique was also mentioned can be used for plasma collection from donors for which blood is withdrawn from the donor's vein by a peristaltic pump through a needle. More recently, few devices were designed to adapt microfluidics or cross-flow filtration chips for separating small volumes of blood (10-100 μl) (7, 8).

The device described in this invention relies on tangential flow filtration (TFF) technology to separate blood cells from serum/plasma component. TFF is a cross-flow filtration process widely used in laboratory and industrial settings to separate or filtrate materials by size exclusion without clogging up the membrane. Although the use of either gravity or pump assisted cross-flow filtration for blood fractionation has been described in patents and publications, there is currently no commercially available device for the most common blood-draw volume of 4-10 ml with vacuum packed tubes; the blood fractionation within this volume range is still mainly done by centrifugation process in the clinical laboratory and yet this blood draw volume range is the most commonly ordered in the clinic. Additionally, innovation of this device is the modular design to keep both plasma/serum and cells for downstream applications; the ease of operation, adaptability of this device to current blood drawn devices and can be easily used at bedside. The uniformity of single step plasma/blood cells separation by the present invention provides a standardized plasma/blood cell collection that is not possible with current multi-step process.

The basic concept of applying TFF to filtrate blood for various purposes has been proposed and is currently in use, mostly for large-scale filtration. A device used directly for routine small-scale blood collection in the range of 4-10 ml has not been described or in commercial use, this device represents a first. This device is advantageously designed to perform a process that currently requires multi-step manual-driven procedures. Additional novelty of this invention is the modular concept for which requires minimum personnel handling, thus is adaptable to downstream automation process. The one device process allows immediate separation of serum or plasma and blood cells for further analysis right at the bedside. Design and hands free processing protect clinical personnel from blood handling hazard. A standardized device ensures uniformity of the results and thus strongly advantageous over the current manual processing methods.

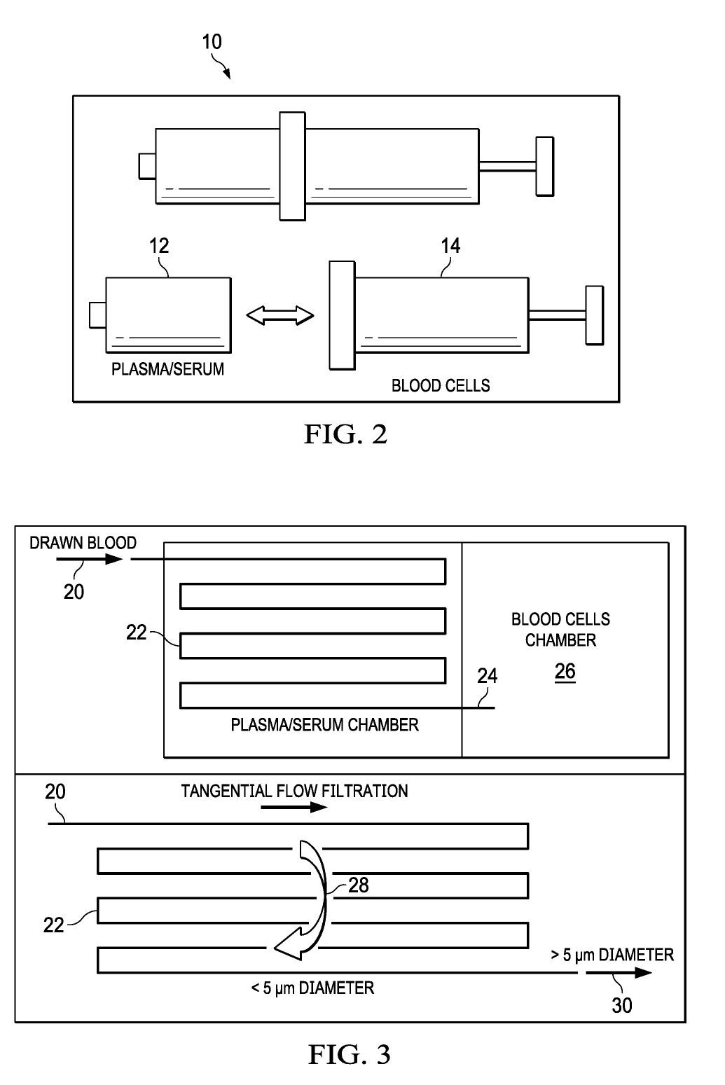

The device as shown in FIG. 2 is one example of the device 10 of the present invention, which can be disposable. The device 10 includes two basic components; one component is the serum/plasma collection chamber 12 contains a TFF unit for serum/plasma, the second component 14 is a collection chamber for blood cells. Typically, the TFF is wetted prior to packaging, that is, it is packaged in a wetted state. By pre-wetting the TFF unit, the TFF unit will operate more consistently, will operate without the need to provide (or have available) a wetting agent, and/or is ready to use without any new or additional steps by the operator. In general, the present invention is intended to be used by the skilled phlebotomist without any change in standard operating procedures and/or without the need for any additional training on the use of the device. The present invention was designed to operate with existing blood collection kits, connect to the same, and follow the same flow as standard phlebotomy without the need for additional equipment such as refrigerators, centrifuges, etc. Two components 12, 14 can be easily separated by a simple twist. The device 10 is designed to fit onto a blood transfer unit via, e.g., a Luer Lock fitting to transfer blood from conventional blood collection tubes. Alternatively, the device 10 could be equipped to include a plastic or rubber cap that fits into a blood collection set for collecting blood directly from subject. The device 10 is designed to be in cylindrical shape similar to standardized blood collection tubes. The device 10 is designed with dimension and shape to accommodate standardized blood transfer unit or blood collection set. The blood fractionation process can simply be accomplished by a slow or timed pull of the plunger unit (˜1 minute), plus a short back flush at the end of operation. The timed pull could either be manual by the person who performs venipuncture and blood collection or simple metered mechanism. Alternatively, the second component 14 is a vacuum assisted collection chamber for blood cells, that is generally a 4 ml or 10 ml tube (but can vary in size depending on the required sample size and draw necessary to obtain sufficient blood or plasma), operationally connected to component 12 via needle puncture.

The plasma/serum and blood cell separation is based on the tangential flow filtration process as illustrated in FIG. 3. A plain view of the plasma/serum collection chamber 12 is shown in the top portion of FIG. 3 that contains a tangential flow filtration (TFF) membrane 22 set either in, e.g., a cylindrical shape or in flat bed shape. The TFF membrane 22 can be, e.g., a long interwoven continuous cylindrical membrane or a set of flat bed shape membranes with connections for continuous flow, although other shapes and sizes are known in the art and would be equally applicable. The TFF membrane 22 has an inlet to receive blood 20 from subject or blood collection tube and an outlet 24 leads into the blood cells connection chamber 26. In the bottom portion of FIG. 3, the blood 20 enters the TFF membrane 22, with a permeate 28 separated from the retentate 30.

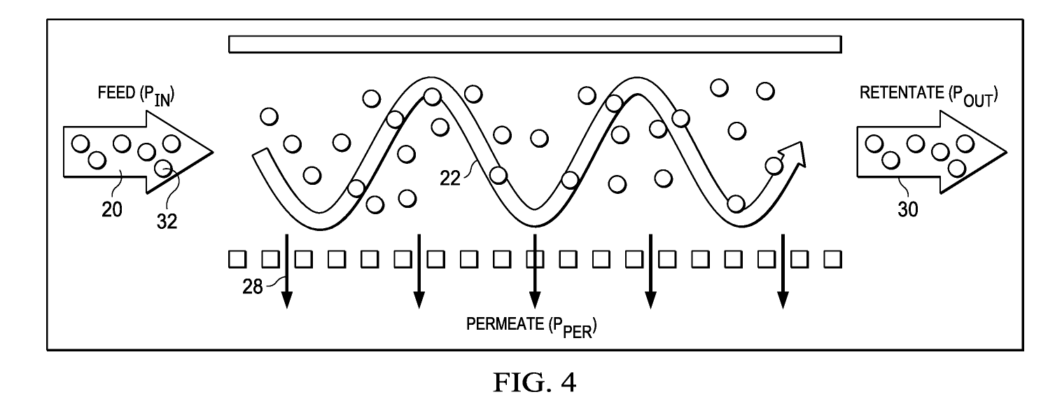

Drawn blood flows through the interior side of the TFF membrane 22 as shown in FIG. 4. FIG. 4 shows a close-up view of the TFF membrane 22, in which blood 20 containing blood cells 32 and plasma enter the TFF membrane 22. The permeate 28 from the TFF membrane 22 is plasma/serum, with some residual fluid containing the blood cells 32 is shown as the retentate 30.

The TFF membrane can be set to have a range of pore size from 0.1 to 10 μm allowing any biological entities less than 5-10 μm in diameter to permeate through membrane into the plasma/serum collection chamber, any biological entities larger than 5-10 μm in diameter (such as blood cells) flow along into the blood cell collection chamber.

In one embodiment, the flow of blood is initiated by the plunger pull in the blood cell collection chamber, the volume of collected blood can be measured by scale on the blood cell collection chamber similar to syringes. Alternatively, the blood drawn can be initiated by suction created with an established vacuum pressure. The blood serum/plasma and cells fractionation process can be accomplished in a minute or so instead of current conventional process requires more than one hour of manual operation.

In another embodiment, the plasma/serum chamber component 12 can be operationally connected in between a blood containing blood connection tube and a vacuum assisted blood collection device to facilitate the plasma/serum and blood cell separation process.

After separation, each chamber can be emptied for further downstream process or stored. Appropriate stabilizing agents and anti-coagulation agents can be placed in each chamber for longer-term storage or the convenience of downstream processing and assays. The individual chamber design for the fractionated blood is novel, each chamber represents a module unit can be easily connected with other modules for further processing. Also, the TFF membrane unit can be changed to contain TFF membrane of varied pore size for additional fractionation.

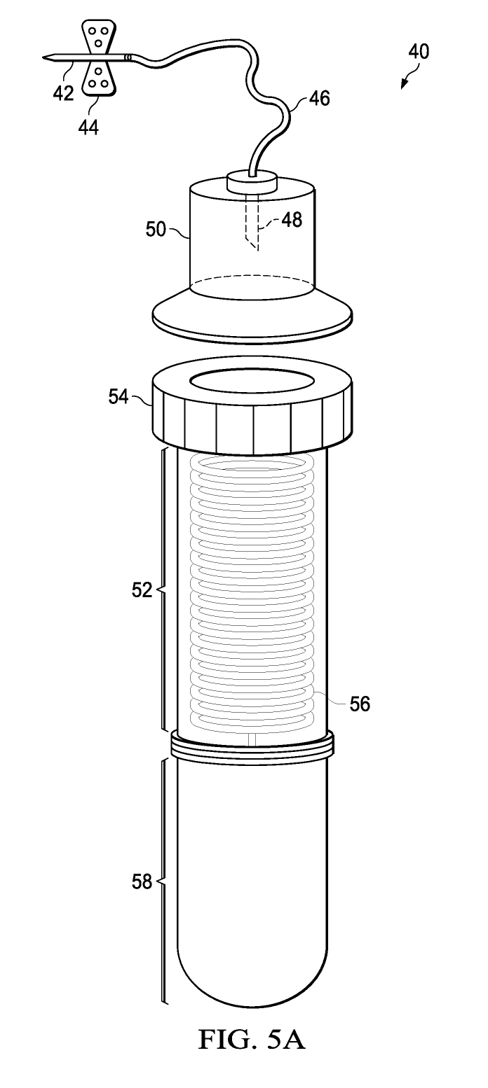

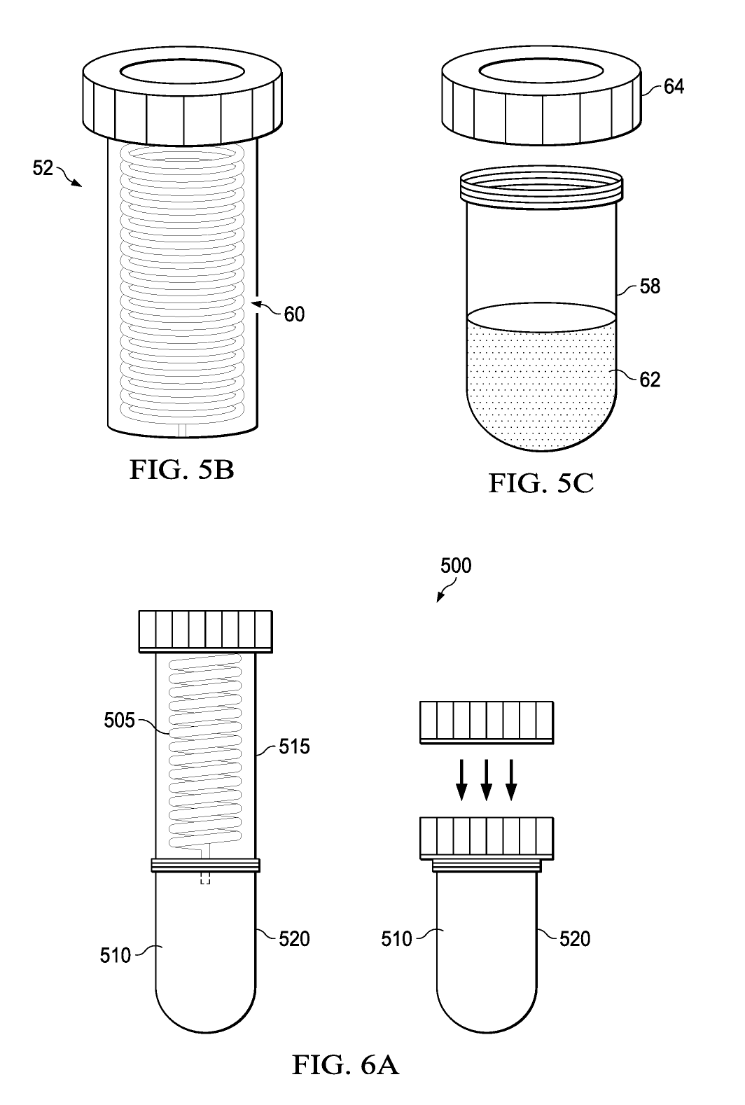

FIGS. 5A to 5C show another embodiment, the TFF membrane unit connects to a blood collection apparatus 40 of the present invention. The blood collection device can be used in conjunction with a standard blood collection needle 42, often connected to a butterfly 44 for ease of use during access to a blood vessel. The needle 42 is connected and in fluid communication with a tube 46 that is connected to a shroud 50 that contains within a needle 48 that helps protect the care provider. The blood collection apparatus 40 of the present invention includes the separation unit 52 and includes the TFF membrane 56 and connects to the needle 48 through a top 54, e.g., a neoprene top commonly used in blood collection units. The separation unit 52 that connects to a blood cell or plasma/serum collection tube 58 that can provide the vacuum necessary to pull the blood through the TFF membrane 56, or an external source of vacuum such as a vacuum line or a syringe can be used to provide the motive force necessary to separate plasma/serum from blood cells through the TFF membrane 56. FIG. 5B shows the separation unit 52 after the blood has been processed and includes within an interstitial space 60 of the separation unit 52 the plasma/serum separated from the blood cells. FIG. 5C shows the blood cell collection tube 58 to which a new top 64, which can be a neoprene top, that can be used for further processing of the cells.

The blood cell collection tube 58 can include a wide variety of components within the tube for preserving the cells or other components. The blood collection tubes can follow and include standard colored tops, labeling or other indicia that indicate the specific type of collection materials or solutions within the collection tube. For example, it is typical for sterile/blood cultures to use, e.g., yellow stopper or bottles. Another example is the use of light blue to indicate a coagulation tube. The skilled artisan will know that if coagulation use and the user uses a butterfly needle, it is necessary to draw a discard tube to collect the air in the tubing into the discard tube, with failure to do so resulting in a short draw which will be rejected by the lab. The present invention compensates for such eventualities by, e.g., increasing the level of vacuum within the tube or from the external source of vacuum. Other indicia include, e.g., red to indicate that non-additive has been included and is a clot activator. If the tube includes a speckled or “tiger” top (or gold) this is a separator tube. To indicate the presence of heparin in the tube, it is common to use, e.g., a green top or indicia. When using a green/gray mottled Plasma Separator Tube (PST) this also includes heparin. Yet another type of tube has a lavender/purple and/or pink top or indicia, which indicates that the tube includes a chelating agent such as EDTA. Finally, it is common to use a gray top or indicia to indicate that the tube includes potassium oxalate and sodium fluoride.

Thus, the present invention can replace the current routine manual-driven operation of blood serum/plasma and cell fractionation in the clinical laboratories. Reliability and hands free process by this device is anticipated to replace the current manual-driven process and gain popularity among clinical operations around the world for an exclusive market.

It is contemplated that any embodiment discussed in this specification can be implemented with respect to any method, kit, reagent, or composition of the invention, and vice versa. Furthermore, compositions of the invention can be used to achieve methods of the invention.

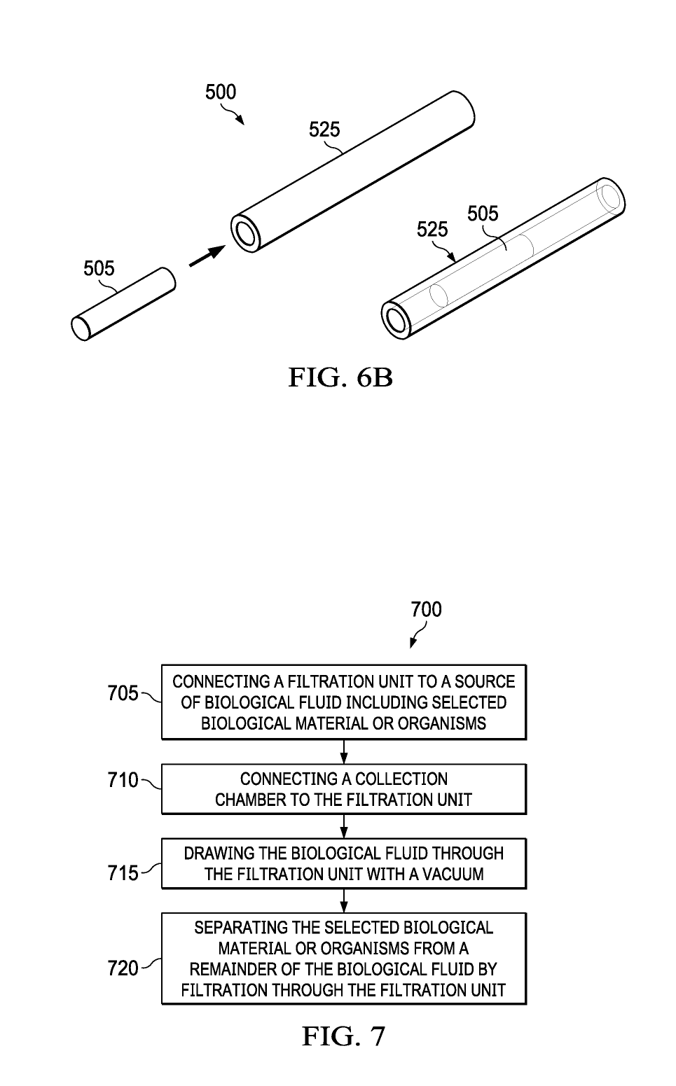

FIG. 6A shows another embodiment of the present invention, a filter apparatus 500. The apparatus 500 may be disposable. The apparatus 500 includes a filtration unit 505 and a collection chamber 510. A biological fluid is drawn through the apparatus and selected organic material (also called “selected organic material” herein) or organisms in the biological fluid are separated from the remainder of the biological fluid. The filtration unit 505 includes one or more hollow fibers.

The filtration unit 505 may be housed in a first tube segment 515 and the collection chamber 510 may be housed in a second tube segment 520, where the first tube segment 515 and the second tube segment 520 are detachably connected by, e.g. a Luer Lock. The filtration unit 505 and the collection chamber may also be housed in a single tube 525 with two open ends of 505 plugged with neoprene caps, as shown in FIG. 6B. The filtration unit is tightly fit inside of 525, and the biological fluid is drawn through the filtration unit 505, where the selected organic material or organisms are retained, and the biological fluid without the selected biological material or organisms flows through the filtration unit 505 to be collected at the bottom of single tube 525. The biological fluid without organisms or the selected biological material can be collected through a draw with syringe and needle through the neoprene cap.

The biological fluid may be drawn into the apparatus 500 by a partial vacuum or a vacuum that is provided by a pump or a syringe (either manually operated or machine-operated). A sample of the biological fluid may have a volume of, e.g., 4-10 mL.

The filtration unit 505 or the collection chamber 510, or both, may include one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, protease/s or salts. The interior surfaces of the filtration unit may be positively charged or negatively charged, and the interior surfaces of the collection chamber may be positively charged or negatively charged.

The hollow fibers of filtration unit 505 may include a mixed cellulose ester, polysulfone, plastic polymers or some combination. In this embodiment, each of the plurality of hollow fibers is held in place relative to the others by an upper tubesheet. Each of the plurality of hollow fibers is open at the upper end held in place by the upper tubesheet, and the biological fluid to be filtered enters the filtration unit 505 at the upper open end. The biological fluid flows along the interior of the hollow fibers, the pores of which are sized to draw off the selected biological material or organisms to be filtered out of the biological fluid, e.g. blood cells. The hollow fibers are closed at the lower ends, i.e., the ends opposite the upper ends held in place by the upper tubesheet, so the selected biological material or organisms drawn off from the biological fluid remains in the interior of the hollow fibers along their lengths but does not exit through their lower ends. Optionally, the lower ends of the hollow fibers may be open and be held in place relative to each other with a lower tubesheet (not shown). The biological fluid drawn into the interior of the hollow fibers flows into the collection chamber 510. When a lower tubesheet is being used, the remainder of the biological fluid will remain in the filtration chamber, depending on the configuration of the lower tubesheet in use. In an embodiment in which the filtration unit 505 and the collection chamber 510 are in separate tubes, the first tube segment 510 can be detached from the second tube segment 520 and the remainder of the biological fluid accessed in the collection chamber 510 in the second tube segment 520. In an embodiment in which the filtration unit 505 and the collection chamber 510 are in a single tube 525, the filtration unit 505 can be removed from the single tube 525 to access the remainder of the biological fluid in the collection chamber 510 at the lower end of the single tube 525.

The hollow fibers of the plurality of hollow fibers may include at least one of a hydrophobic material, a hydrophilic material, or a low-protein-binding material. They may include pore sizes ranging from about 0.5 microns to about 5 microns, depending on the intended use or uses. They may have various densities ranging from 50 to 500. They may have various surface areas ranging from 500 cm² to 3000 cm². The apparatus 500 may operate at various vacuum pressures ranging from −3 psi to −11 psi.

In another embodiment, the plurality of hollow fibers is disposed within the apparatus 500 such that the closed ends of the fibers disposed toward the upper end of apparatus 500 and the open ends of the hollow fibers are at disposed toward the lower end of the apparatus 500, with the lower end of the filtration unit 505 sealed except for the hollow tube openings at the lower end. In this embodiment, the biological fluid is introduced to the apparatus 500 at the upper end, and it flows past the plurality of hollow fibers in the filtration unit 505. A portion of the biological fluid, e.g., plasma, is drawn off into the hollow fibers and continues through the open ends into the collection chamber 510. The selected biological material or organisms, e.g., blood cells, are retained in the filtration unit 505.

In another embodiment, the filtration unit 505 includes two sets of one or more hollow fibers. The first set of one or more hollow fibers is disposed within the filtration unit with open ends toward the upper end of the filter apparatus 500, and the second set of one or more hollow fibers is disposed below the first plurality and with its closed end toward the upper end of the filter apparatus 500. In this embodiment, the biological fluid, e.g., whole blood, is introduced to the apparatus 500 at the upper end and flows past the first set of one or more hollow fibers, which retain, e.g., blood cells, and allow the remainder of the biological fluid, e.g., plasma, to pass through the second set of one or more hollow fibers and into the collection chamber 510.

In one exemplary, non-limiting apparatus 500, such as the one shown in FIG. 6B, the filtration unit 505 and the collection chamber 510 are housed in a single tube 525 that is 16 cm long and with a diameter of 12.7 mm. At the end of the single tube 525 adjacent to the filtration unit, there is a space about 5 mm long to accommodate the end of a needle and biological fluid entering the apparatus 500. In the filtration unit 505, there are 75 mixed cellulose esters hollow fibers, each 10 cm long, with 0.1 micron pores, an outer diameter of about 1 mm and an inner diameter of 0.6 mm, and with a total calculated capture volume of about 2.2 mL. The collection chamber 510 is about 3 cm long, with a volume of about 3.8 mL. The ends of the single hollow tube 525 have neoprene caps that permit adding and withdrawing fluid with syringes. A partial vacuum between −7 and −11 psi is used with this exemplary apparatus 500 to draw the biological fluid into the apparatus 500 and through the filtration unit 505.

A typical use of this exemplary, non-limiting apparatus 500 includes using a syringe to inject blood through the neoprene cap into the 5 mm space adjacent to the filtration unit 505. A vacuum inside of the tube draws the blood from the space into the filtration unit 505. The blood organisms are filtered out there and the remaining serum/plasma flows into the collection chamber 510. The plasma/serum is removed from the collection chamber 510 with a syringe through the neoprene cap at the other end of the tube.

The apparatus 500 may be included in a kit that also includes, e.g., a sample tube, a syringe, gauze, tape and/or other items useful for collection a blood or plasma/serum sample.

Embodiments of the present invention such as the filter apparatus 500 may be included in a filter kit, which may also include items commonly used during a phlebotomy procedure, such as gauze, tape, needles, etc.

FIG. 7 illustrates a flowchart for a method 700 of filtering a biological fluid. Block 705 includes connecting a filtration unit to a source of biological fluid including selected biological material or organisms. Included in block 710 is connecting a collection chamber to the filtration unit. Block 715 includes drawing the biological fluid through the filtration unit with a vacuum. Separating the selected biological material or organisms from a remainder of the biological fluid by filtration through the filtration unit is included in block 720.

Testing of embodiments of the present invention has been performed as described herein.

### MATERIALS

Human whole blood: Donor Human whole blood was obtained from Discovery Life Sciences (Los Osos, Calif.) and Innovative Research (Novi, Mich.) with IRB approval for research purpose. Donor Human Whole Blood was drawn from healthy donors in FDA-licensed facilities. All lots have been tested by FDA-approved for Human Immunodeficiency Virus RNA (HIV-1 RNA), Antibodies to Immunodeficiency Virus (Anti-HIV 1/2), Antibodies to Hepatitis C Virus (HCV), Hepatitis C Virus RNA (HCV RNA), Hepatitis B Virus (HBV DNA), Hepatitis B Surface Antigen (HbsAg), and Syphilis. Whole blood was collected with 10 ml K2EDTA VACUTAINER® by BD and shipped with cold pack overnight via FedEx.

Cultured cells: HL-60 cells human leukemia cell line was purchased from ATCC and cultured in RPMI media supplemented with 10% bovine serum.

Chemicals and reagents from various manufacturers were purchased through Quartzy.com. Pre-cast E-gels were purchased from ThermoFisher.

Fabricated tangential flow filtration cartridges and hollow fiber membranes were provided by Koch Membrane Systems (Wilmington, Mass.) and Spectrum Labs/Repligen Corp. (Rancho Dominguez, Calif.).

Tangential flow filtration units were also constructed in 16 mm×100 mm VACUTAINER® blood collection tubes. Hollow fibers were constructed in one tubesheet and one sealed end format (C F Wan, T Yang, G G Lipscomb, D J Stookey, T S Chung. Design and fabrication of hollow fiber membrane modules. Journal of membrane science 538, 96-107). The open lumen ends of hollow fibers were embedded in the tubesheet, and the sealed ends of the fibers were free and sealed with epoxy glue (Gorilla Glue). The hollow fiber bundles in the tubesheet were tightly fitted into the neoprene cap of the VACUTAINER®. Vacuum pressure in the filtration test unit was established with a hand-operated vacuum pump through a 22 G needle.

### METHODS

Plasma isolation: Whole blood collected in K2EDTA VACUTAINER® was stored at 4° C. before processing for plasma. Whole blood was first centrifuged at 1400 rcf for 12 minutes. The upper plasma layer was transferred to a fresh 15 ml conical tube without disturbing the cell layer. The transferred plasma was centrifuged again at 1400 rcf for 12 minutes, and after the second centrifugation the upper layer was removed as a plasma fraction for comparative analysis.

DNA isolation: DNA from plasma was isolated using QIAamp MinElute ccfDNA kit (Qiagen, Germantown, Md.) by following manufacturer-recommended procedures. The isolated DNA was quantified using Take 3 plate of BioTek Synergy H1 plate reader.

Hemoglobin Analysis: The presence of hemoglobin in samples was measured by spectrophotometer at OD 414 with BioTek Synergy H1 plate reader. A standard curve was established with a dilution of a known amount of hemoglobin in bovine serum.

Glyceraldehyde 3 Phosphate Dehydrogenase (GAPDH) Assay: GAPDH was detected by using Human GAPDH ELISA Kit (mybiosource.com) by following the manufacturer-recommended procedures. The ELISA plate was read with BioTek Synergy H1 plate reader. The ELISA kit has the detection range from 0.5-10 ng/mL. High concentration samples were diluted to fit the standard curve.

DNA analysis by agarose gel electrophoresis: Purified DNA was analyzed on a pre-casted E-Gel system (Invitrogen). 100 bp ladder was purchased from Invitrogen.

### RESULTS

### EXAMPLE 1

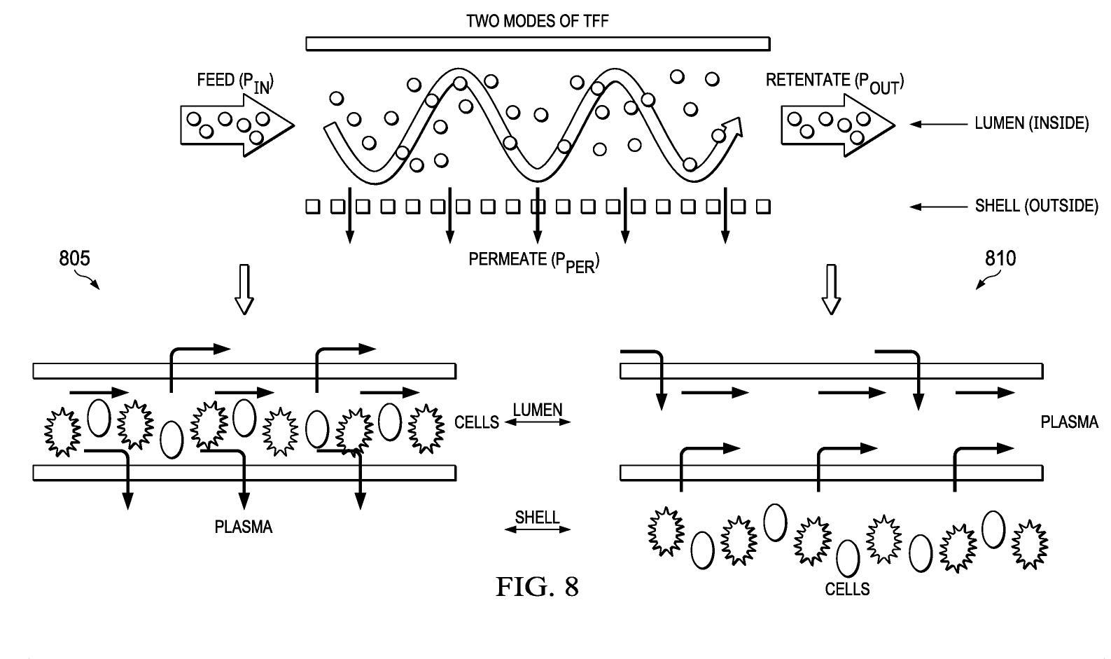

There are currently no vacuum based (i.e. VACUTAINER®) blood collection tubes that can readily separate the plasma from blood cells of the whole blood. Although hemodialysis routinely utilizing hollow fiber tangential flow filtration technology for blood toxin removal and the application of such technology is continuing to improve (Ronco C, Clark W R. Haemodialysis Membranes. Nat Rev Nephrol. 2018 June; 14(6):394-410), it has not been clear until the present invention whether the similar tangential flow hollow fiber format is applicable to isolate plasma from the whole blood by a vacuum assisted process. In addition, the hemodialysis units were designed to filter away the small molecular weight toxins in a continuous flow process rather than obtain the plasma fraction in a single step, but the effectiveness and operability has not been clear until the present invention. The hemodialysis process flows the whole blood through the lumen of the fiber and clear the filtrate from the shell space in an inside-out process, as illustrated in FIG. 8, which depicts two modes of tangential flow filtration. The inside-out mode 805 is to flow whole blood through the lumen of the fiber, with the plasma to filter out to the shell space. The outside-in mode 810 is to flow whole blood through the shell space of the fiber bundle, with the plasma to filter into the lumen of the fiber bundles. For the intended objective of this invention to obtain plasma from blood cells, the hollow fibers will need to serve either as a collection pouch in an inside-out mode 805 (retaining blood cells in the lumen of the hollow fibers) or as an extraction device in an outside-in mode 810 (retaining blood cells in the shell space of hollow fiber unit). Neither process or design has been reported.

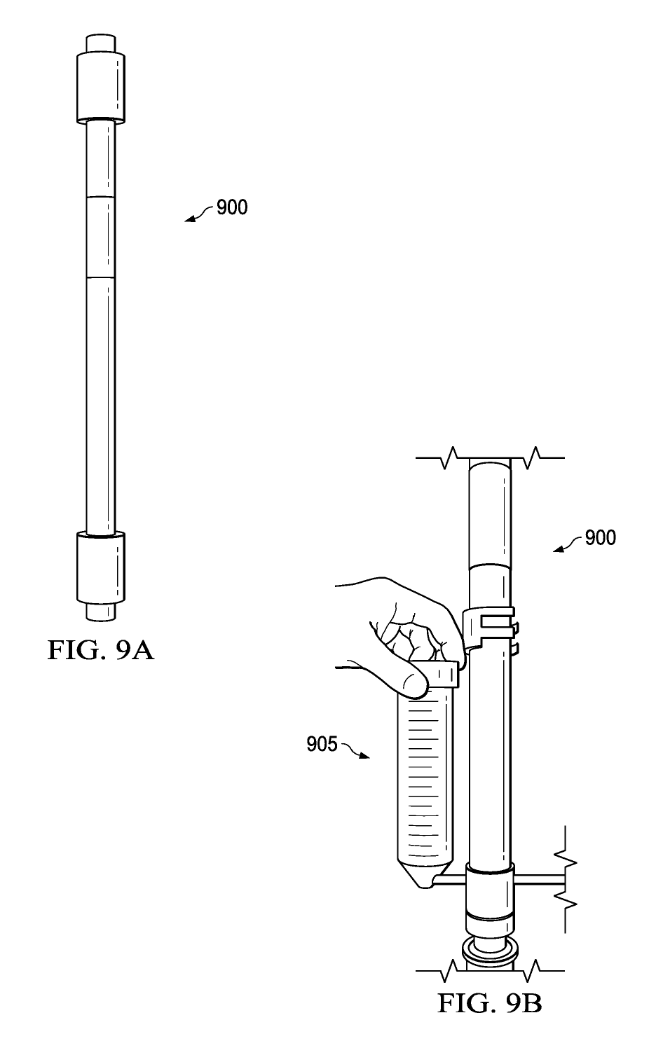

To test the applicability of obtaining plasma in either an inside-out mode 805 (whole blood enters through lumen of the fibers) or an outside-in mode 810 (whole blood enters through shell space), pre-fabricated tangential flow hollow fiber filtration cartridges similar to hemodialysis units were tested to separate plasma from blood cells in the whole blood. Such a cartridge 900 is shown in FIGS. 9A and 9B. FIG. 9A pictures a tangential flow filtration (TFF) cartridge 900. FIG. 9B pictures the TFF cartridge 900 filled with whole blood (on the right) and collected plasma filtrate fractions pooled into a 50 ml conical tube 905 (on the left). The cartridges 900 were 1″ in diameter and 18″ long, smaller than hemodialysis units but with a similar two-tubesheets format. The test cartridges 900 were fully packed with polysulfone (PS) membrane hollow fibers with 0.5 micron performance pores. The 0.5-0.6 micron performance pore size could be the most optimum pore size required to obtain important biologicals from the blood without blood cells. Typical hemodialysis units have much smaller pore size (around 10 kb molecular weight cut-off (MWCO)) to filter away small toxin molecules. Top and bottom ports allow fluids passing through the lumen of the fibers, and two side ports allow permeate filtrate collection or removal of filtrate from the shell space. The unit was first rinsed extensively with house distilled water by pumping it through the shell space via the two side ports and through the lumen via the top and bottom ports. After an extensive rinse, the unit was primed with PBS before use.

The inside-out mode 805 was tested first. After priming, human whole blood was pumped through the lumen of the units through the top port of the cartridge 900 shown in FIGS. 9A and 9B. The red blood cells can be readily observed entering through the lumen and spreading downward through the fiber bundle. The spread of red blood cells is rather uneven: some fibers are faster than others, possibly due to uneven fiber packing and entry through tubesheet at the top of the cartridge 900. As the blood continues to enter through the cartridge 900, the fluids (plasma+PBS) flowing out of the bottom side port became opaque, indicating a plasma-like permeate from the filtration process. The permeate was collected in 2 ml fractions and analyzed.

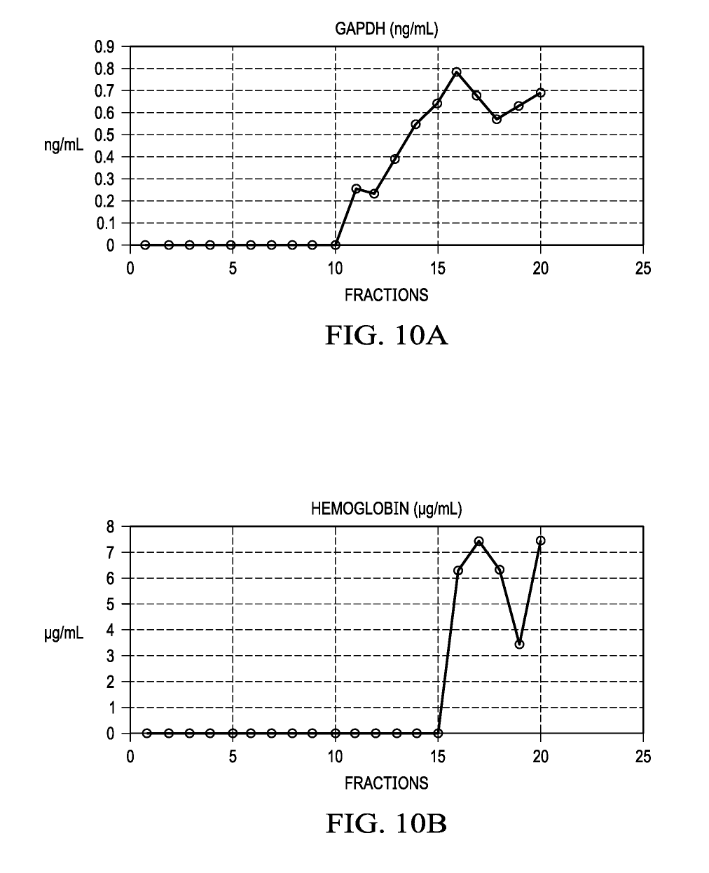

The bed volume of the cartridge 900 was about 40 ml, the whole blood was initially pumping through at 15 ml per minute, and the initial permeate was a straw-like color. When the pump speed was gradually increased to 45 ml per minute, the permeate flowing out began to turn pinkish, indicating potential hemolysis of red blood cells. Each collected fraction was assayed for GAPDH and hemoglobin, the data for which is shown in FIGS. 10A and 10B. The graph of FIG. 10A shows the GAPDH protein concentration in each fraction. The graph of FIG. 10B shows the hemoglobin concentration in each fraction. The data points represent averages of three analyses. The baseline of zero represents samples below the limitation of detection.

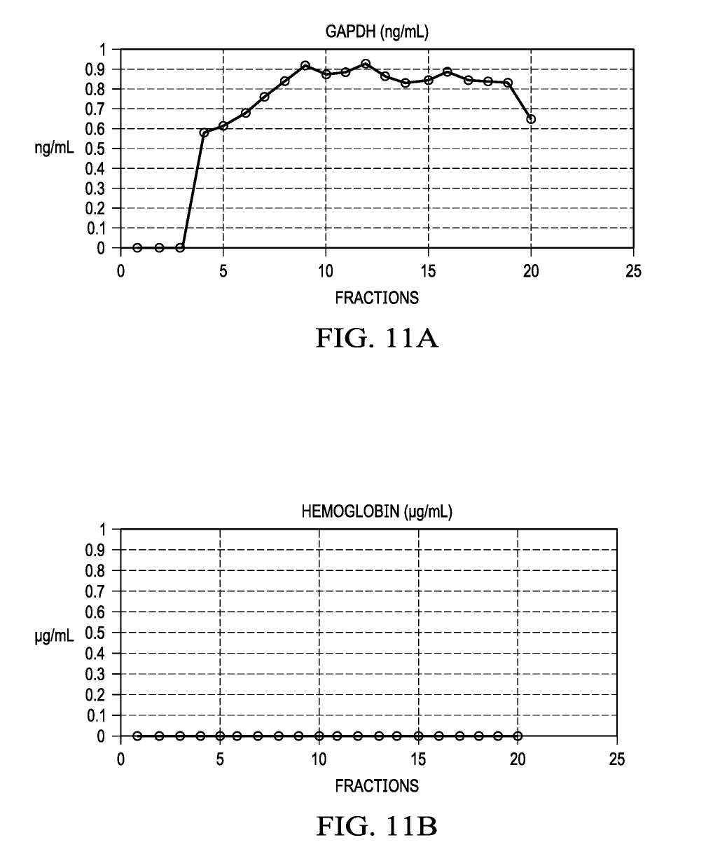

The outside-in mode 810 was tested next. After priming, human whole blood was pumped into the shell space of the unit through the bottom side port, initially at 15 ml per minute. The unit was evenly distributed with blood cells, as red was visibly spreading slowly upwards through the unit. Unlike the inside-out mode 805, the outside-in mode 810 was spread evenly throughout the cartridge 900. The pumping speed was gradually increased to 45 ml per minute. A slightly opaque filtrate was continuously dripped from the bottom port with a plasma-like appearance after pooled fractions together in the conical tube in FIG. 9B). Increased pump speed did not increase the pink appearance, indicating less hemolysis of red blood cells or no hemolysis. Each collected fraction was assayed for GAPDH and hemoglobin, the data for which is shown in FIGS. 11A and 11B. The graph of FIG. 11A shows the GAPDH protein concentration in each fraction. The graph of FIG. 11B shows the hemoglobin concentration in each fraction. The data points represent averages of three analyses. The baseline of zero represents samples below the limitation of detection.

GAPDH was selected for detection of plasma protein in samples because GAPDH is not as highly abundant as albumin, so it is more sensitive in detecting recovery efficiencies using GAPDH as the marker. The lower limit of detection of GAPDH protein for the ELISA assay kit was 0.1 ng/mL. Reasonable amount of plasma protein was recovered from the filtrate fractions. For the inside-out mode 805, the plasma protein was not detected until fraction #11. In contrast, GAPDH protein was detected as early as fraction #4 with the outside-in format 810 (see FIG. 11A). The outside-in mode 810 also obtains a higher concentration of GAPDH than the inside-out mode 805 (See FIG. 11A). The difference could be from the direction of movement of the samples. It was noticed that the sample fed from the top was not as evenly distributed as a sample fed from the bottom. In both cases, the plasma protein was effectively recovered without an apparent presence of blood cells in the filtrate. (Each fraction was centrifuged, and no cell pellets were observed.) Increased speed showed a slight increase of hemoglobin in the filtrate of the inside-out mode 805 (see FIG. 10B); on the other hand, hemolysis was not detected with the outside-in mode 810 within the limitation of the assay method (See FIG. 11B). In conclusion, the tangential flow filtration process is feasible for separating blood cells from plasma with either the inside-out mode 805 or the outside-in mode 810, and the outside-in mode 810 may have a better performance. Using the present invention, plasma can be effectively separated from blood cells and recovered without significant hemolysis. The direction of flow of whole blood and speed of flow could be key influencing factors.

### EXAMPLE 2

The test with large units reported above strongly indicated the feasibility of utilizing tangential flow filtration hollow fibers to obtain plasma from the whole blood. The application could be with either inside-out mode 805 or outside-in mode 810. For the purpose of the present invention, much smaller units are required for bed-side blood collection operations. Additionally, instead of an open-end lumen for whole blood to flow through, the unit of this invention will need to have sealed end hollow fibers to either capture the blood cells in the lumen (inside-out mode 805, FIG. 8) or in the shell space (outside-in mode 810, FIG. 8) in a one tubesheet and one sealed end format (C F Wan, T Yang, G G Lipscomb, D J Stookey, T S Chung. Design and fabrication of hollow fiber membrane modules. Journal of membrane science 538, 96-107).

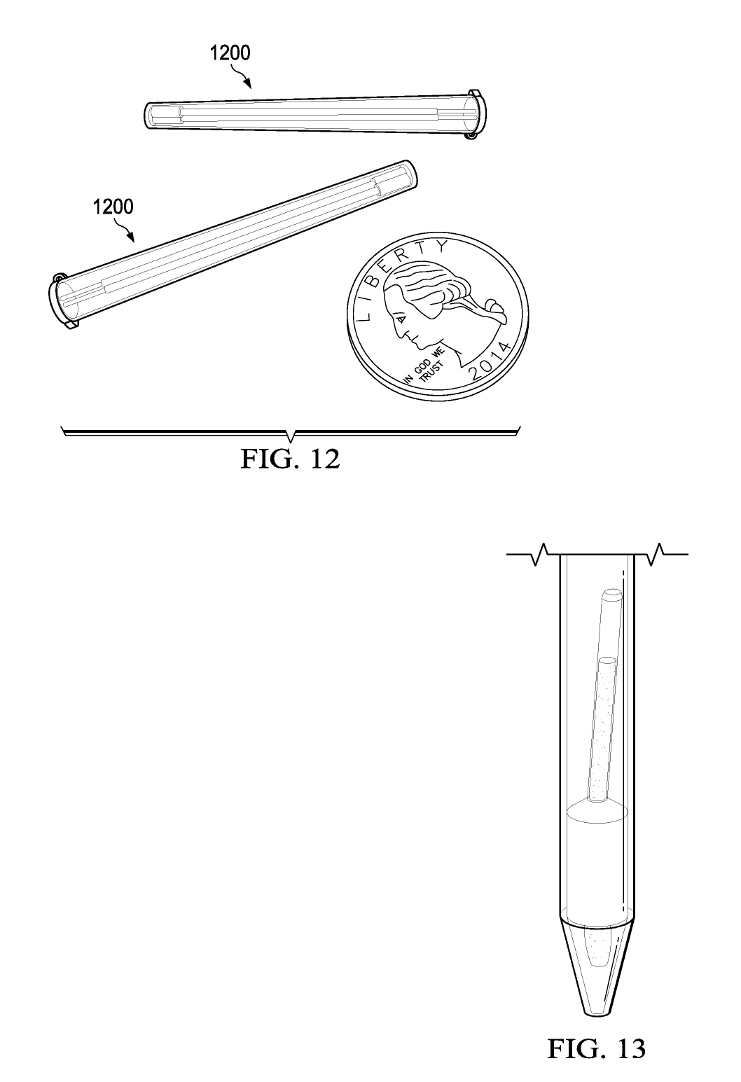

To further test this potential, small and simple test units 1200 were made by Spectrum Labs. The small units are shown in FIG. 12. Each unit 1200 consists of a single loop of hollow fiber, made of Spectrum's modified polyether sulfone (mPES) membrane with a 0.65 micron pore size. The tubesheet construct at the bottom had two lumen openings. The inner diameter of the fiber was 0.75 mm and the wall of the fiber was 0.2 mm thick. The outer diameter of the housing was 5 mm with a 4-mm inner diameter, and the housing was 4.5 cm in length from the top to the tubesheet. The top was opened with the single hollow fiber bent at the top to fit into the housing. The shell space was wide open all the way to the top. Material could be loaded into the shell space from the top and filtrate collected from the lumen openings at the bottom. The fill volume was 400 μl.

When the test unit 1200 was filled by putting either water or PBS into the shell space, fluids dripped freely from the lumen exit openings. However, when the shell space was filled with whole blood, no flow or very slow flow by simple gravity was observed. Next, the test device was loaded with whole blood and then a force exerted either by syringe push or by centrifugation pull; with exerted force, approximately 200 μl of filtrate out of a total 360 μl of whole blood was able to pass through, but the filtrate was reddish, indicating hemolysis, as shown in FIG. 13. Out of a total 360 μl whole blood loaded, 200 μl of filtrate represents about 55% of total volume, which is consistent with the percentage of plasma volume in whole blood. The initial attempt of centrifugation was with a swing bucket clinical centrifuge at 500 rpm (˜106×g) for 10 minutes. When the centrifugation speed was lowered to the lowest instrument speed of 200 rpm (˜17×g) for 10 minutes, the same amount of filtrate was collected, and the filtrate was still reddish.

The initial tests with this small unit 1200 indicate centrifugation force was able to push the plasma through with comparable amount of filtrate (plasma) collected through the filtration, but the reddish filtrate indicates hemolysis. Although the filtration process appears to be efficient, the hemolysis indicates potential cell breakage. The test units 1200 have only two strands of fibers, so there may not be sufficient surface area for efficient flow filtration. The centrifugation force might be too strong, resulting in compacting all cells at the bottom of the unit 1200 (see FIG. 13, the test unit in a 15 ml conical tube); this testing format was more like dead-end filtration rather than tangential flow filtration. The GAPDH and hemoglobin level of each test is summarized in table 1.

TABLE 1
Summary results of small filtration unit. Results with
whole blood loaded directly into the small filtration unit.

| | Whole | | | |
|---|---|---|---|---|
| | blood | Filtrate | GAPDH | Hemoglobin |
| Conditions | Load | Recovered | (ng/mL) | (μg/mL) |
| Plasma | | | 0.88 | 0 |
| Centrifuge 106xg | 360 μl | 200 μl | 0.8 | 55 |
| syringe push | 360 μl | 210 μl | 0.76 | 98 |
| Centrifuge 17xg | 360 μl | 200 μl | 0.85 | 48 |

Note to Table 1: Plasma is a plasma sample obtained by the conventional double centrifugation method for comparison. A zero indicates sample below the limitation of detection.

Next, the whole blood was held in a 1-mL syringe with syringe connected to the top of the test filtration unit; this configuration was to allow the whole blood to flow into the filtration unit to initiate tangential flow rather than compacted dead-end filtration. With the same volume of 360 μl whole blood loaded and spun at the lowest speed of 200 rpm, the same amount of filtrate was recovered and the filtrate had much less red color strongly indicating less hemolysis. This test format was further explored by loading a smaller volume of whole blood. When loading 150 μl of whole blood, the hemoglobin level was below the sensitivity of detection. The result is presented in table 2.

TABLE 2
Summary results of small filtration unit. Results with
whole blood load on top of the small filtration
unit to allow blood to flow through the filtration unit.

| | Whole | | | |
|---|---|---|---|---|
| | blood | Filtrate | GAPDH | Hemoglobin |
| Conditions | Load | Recovered | (ng/mL) | (μg/mL) |
| Plasma | | | 0.88 | 0 |
| Centrifuge 17xg | 360 μl | 200 μl | 0.82 | 26 |
| Centrifuge 17xg | 150 μl | 80 μl | 0.89 | 0 |

Note to Table 2: Plasma is plasma sample obtained by the conventional double centrifugation method for comparison. A zero indicates sample below the limitation of detection.

The tests with the small simple filtration test unit again demonstrated that the outside-in mode 810 is feasible to separate plasma from blood cells. Unfortunately, because of the small openings for the lumen port, it was not possible to demonstrate the inside-out mode 805. The small test units were much rougher for blood cells: although there was significant amount of plasma being filtered out, the hemolysis was also apparent (see Table 1). The high levels of hemolysis may be due either to the centrifugation force or to the strong-push to accelerate the filtration process. Also, with pre-filled test units, the process is more like the dead-end filtration process than the tangential-flow filtration process. Indeed, when the whole blood was allowed to flow into the filtration unit, the hemolysis was greatly reduced, particularly when smaller samples were applied (see Table 2). In conclusion, the tangential-flow filtration is applicable in smaller units, but the flow of whole blood, the applied force and the surface area need to be well designed and calculated.

### EXAMPLE 3

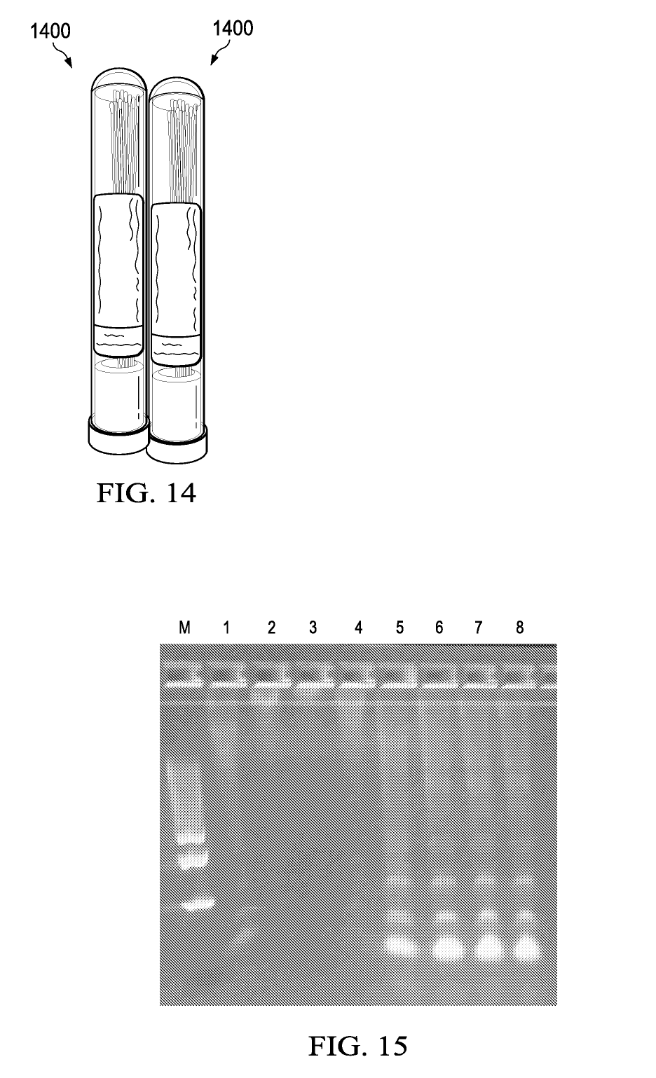

To test the performance of filtration units under vacuum conditions, test filtration units were constructed in conventional BD blood collection tubes 1400, pictured in FIG. 14 and described in the materials and methods description above. A vacuum was established and whole blood was delivered into the vacuum tube through the neoprene cap by a 22 G needle-attached syringe. The lumen of the hollow fibers was open at top and dead-ended at the bottom to allow whole blood cells to be captured inside the lumen and plasma to be filtered out into the bottom of the tube 1400. Two types of hollow fibers membranes were tested; one is mixed cellulose ester (ME) with a 0.1-micron pore and another one is polysulfone (PS) with 10K MWCO. PS test units have 30 strands of hollow fibers in each unit, and ME test units have a range of 25 to 50 strands of hollow fibers in each unit 1400.

One ml of whole blood was used to test each unit. Two vacuum pressures were tested; a lower pressure of approximately 200 mmHg and a higher pressure of approximately 300 mmHg). Most test units obtained plasma successfully without blood cell contamination, but a few test units had blood cells in the plasma due to the leakage through tubesheet. Whole blood used for this set of experiment was from a single female 70-year-old donor in a single batch. The data is summarized in table 3.

TABLE 3
Summary results of tests with constructed vacuum-packed filtration units.

| | | | Whole blood | Filtrate | | | |
|---|---|---|---|---|---|---|---|
| Sample | Filtration Unit | Vacuum Applied | Load | Recovered | GAPDH (ng/mL) | Hemoglobin (μg/mL) | Note |
| Control | Plasma | | | | 0.88 | 0 | |
| 1 | PS, 30 fibers | 300 mmHg | 1 ml | 900 μl | 1.6 | 98 | Leak |
| 2 | PS, 30 fibers | 300 mmHg | 1 ml | 600 μl | 0.76 | 12 | |
| 3 | PS, 30 fibers | 200 mmHg | 1 ml | 580 μl | 0.85 | 0 | |
| 4 | ME, 30 fibers | 300 mmHg | 1 ml | 820 μl | 1.34 | 58 | Leak |
| 5 | ME, 30 fibers | 200 mmHg | 1 ml | 950 μl | 1.8 | 89 | Leak |
| 6 | ME, 35 fibers | 200 mmHg | 1 ml | 450 μl | 0.96 | 0 | |
| 7 | ME, 40 fibers | 200 mmHg | 1 ml | 450 μl | 0.9 | 0 | |
| 8 | ME, 50 fibers | 200 mmHg | 1 ml | 580 μl | 0.93 | 0 | |
| 9 | ME, 50 fibers | 200 mmHg | 1 ml | 600 μl | 0.88 | 0 | |

Note to Table 3: Plasma is plasma sample obtained by the conventional double centrifugation method for comparison. A zero indicates sample below the limitation of detection.

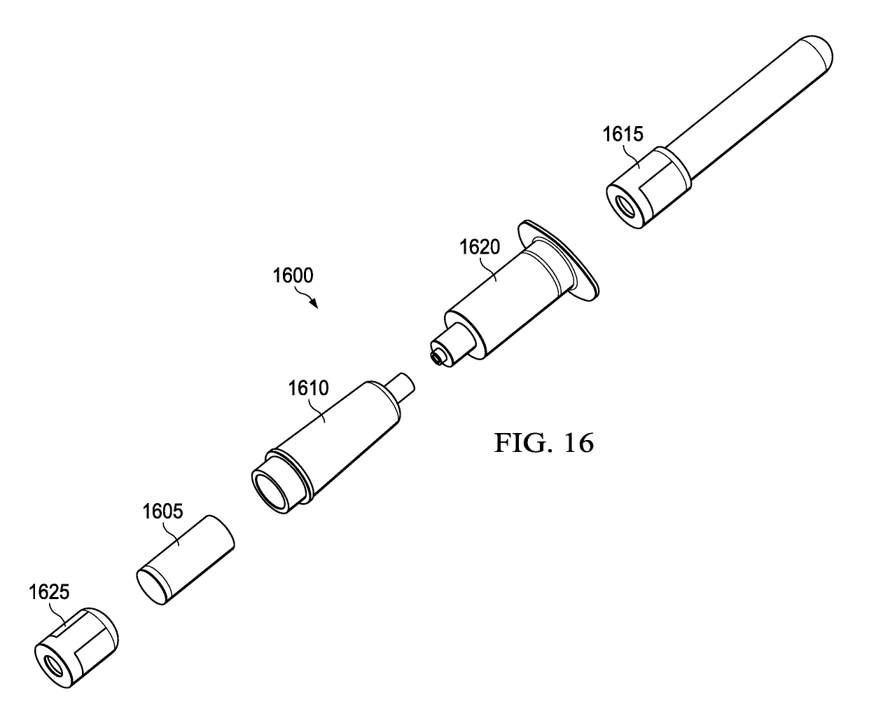

Collected plasma samples were extracted for nucleic acids, and a plasma sample obtained with the standard two-centrifugation procedure was also extracted for comparative purposes. An equal amount of nucleic acids was run onto a 4% agarose gel. The gel electropherogram is shown in FIG. 15.

This set of experiments demonstrate that vacuum force is able to pull whole blood through the filtration unit. Due to imperfection of construction, a few units showed leakage. Leakage is evident from higher filtrate volumes that were collected with stronger presence of hemoglobin in collected samples (see Table 3, samples #1, 4 and 5). Nucleic acids isolated from the leaked samples show mostly high molecular weight genomic DNA (See FIG. 15, lanes 2, 3 and 4). In contrast, successfully filtrated plasma samples showed a significant and distinguished pattern of cell-free DNA with a background of nucleic acids (see Table 3, samples #6, 7, 8 and 9; and FIG. 15, lanes 5, 6, 7 and 8, respectively). Samples from PS units yielded a very low concentration of nucleic acids, possibly due to the low MWCO of the hollow fiber, there was not a sufficient concentration of nucleic acids on the gel to be analyzed.

Nucleic acids isolated from the plasma obtained through the constructed novel filtration units showed significant recovery of smaller nucleic acids with distinctive cell-free DNA pattern. The yield of cell-free DNA (see FIG. 15, lanes 5-8) appears to be more intense with less large molecular weight genomic DNA at the top of the gel when compared with nucleic acids isolated from plasma processed through the conventional two-centrifugation method (see FIG. 15, lane 1). All samples were from the same batch of a single donor.

In summary, the novel blood collection device and method of the present invention provide strong advantages over the current method of isolating plasma from the whole blood. The novel device was shown to be easier, speedier and efficient to operate than the current plasma isolation process. Nucleic acids isolated from plasma obtained through this invention device are better in quality with less high molecular genomic DNA contamination (a crucial factor for nucleic acid-based liquid biopsy (Elazezy M, Joosse S A. Techniques of using circulating tumor DNA as a liquid biopsy component in cancer management. Comput Struct Biotechnol J. 2018 Oct. 9; 16:370-378)). A test unit 1600, as shown in the computer assisted design (CAD) diagram in FIG. 16, can be readily made to initiate clinical testing. A hollow fiber bundle 1605 in tubesheet and dead-end format is to be fit into a housing 1610, the housing 1610 is to be connected with a VACUTAINER® 1615 via blood transfer unit 1620, and a neoprene cap 1625 is to be used to facilitate whole blood entry with vacuum draw by VACUTAINER® 1615. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112 as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

(1) Volik S, Alcaide M, Morin R D, Collins C. Cell-free DNA (cfDNA): Clinical Significance and Utility in Cancer Shaped By Emerging Technologies. Mol Cancer Res. 2016 October; 14(10):898-908.

(2) Elshimali Y I, Khaddour H, Sarkissyan M, Wu Y, Vadgama J V. The clinical utilization of circulating cell free DNA (CCFDNA) in blood of cancer patients. Int J Mol Sci. 2013 Sep. 13; 14(9):18925-58.

(3) Jiang P, Lo Y M. The Long and Short of Circulating Cell-Free DNA and the Ins and Outs of Molecular Diagnostics. Trends Genet. 2016 June; 32(6):360-71.

(4) Thatcher S A. DNA/RNA preparation for molecular detection. Clin Chem. 2015 January; 61(1):89-99.

(5) Gurland, H. J., Samtleben, W., and Blumenstein, M., 1983, Therapeutic membrane plasmapheresis, Present state and future aspects. Life Support Systems, 1:61-70.

(6) Solomon, B. A., Castino, F., Lysaght, M. J., Colton, C. K., and Friedman, L. I., 1978, Continuous flow membrane filtration of plasma from whole blood, Trans. Am. Soc. Artif. Int. Organs 24:21-26.

(7) Homsy A, van der Wal P D, Doll W, Schaller R, Korsatko S, Ratzer M, Ellmerer M, Pieber T R, Nicol A, de Rooij N F. Development and validation of a low cost blood filtration element separating plasma from undiluted whole blood. Biomicrofluidics. 2012 March; 6(1): 12804-128049.

(8) Yang S, Undar A, Zahn J D. A microfluidic device for continuous, real time blood plasma separation. Lab Chip. 2006 July; 6(7):871-80.

What is claimed is:

1. An apparatus comprising:
a wetted tangential flow filtration unit capable of separating the cells from a biological fluid by tangential flow filtration; and
a vacuum-assisted collection chamber connected to the tangential flow filtration unit, wherein a vacuum draws the biological fluid from the source through the tangential flow filtration unit to separate the cells from the biological fluid.

2. The apparatus of claim 1, wherein the vacuum-assisted collection chamber collects the cells, and the wetted tangential filtration unit collects a cell-free fluid or a cell-free fluid and the wetted tangential filtration unit collects cells.

3. A method of separating cells from a biological fluid comprising:
connecting a wetted tangential flow filtration unit to the source of biological fluid comprising cells and capable of separating the cells from the biological fluid by tangential flow filtration;
connecting a vacuum-assisted collection chamber to the wetted tangential flow filtration unit, wherein a vacuum draws the biological fluid from the source through the tangential flow filtration unit to separate the cells from the biological fluid; and
separating the cells from the biological fluid by tangential flow filtration into a retentate comprising the cells and a permeate that comprises a cell-free fluid.

4. The method of claim 3, wherein the vacuum-assisted collection chamber collects the cells, and the wetted tangential filtration unit collects a cell-free fluid or collects a cell-free fluid, and the wetted tangential filtration unit collects cells.

5. polyanetholepolyanetholeA kit comprising:
a wetted tangential flow filtration unit capable of separating the cells from a biological fluid by tangential flow filtration; and
a vacuum-assisted collection chamber connected to the tangential flow filtration unit, wherein a vacuum draws the biological fluid from the source through the tangential flow filtration unit to separate the cells from the biological fluid.

6. A filter apparatus comprising:
a filtration unit; and
a collection chamber in fluid communication with the filtration unit;
wherein a vacuum draws the biological fluid through the apparatus; and
wherein selected biological material comprised by the biological fluid is separated from a remainder of the biological fluid.

7. The apparatus of claim 6, wherein the selected biological material is substantially retained by the filtration unit and the remainder of the biological fluid is substantially passed to the collection chamber or the selected biological material is passed to the collection chamber and the remainder of the biological fluid is substantially retained by the filtration unit.

8. The apparatus of claim 6, wherein the filtration unit comprises a hollow-fiber filtration unit, a membrane filtration unit, or a cassette filtration unit.

9. The apparatus of claim 6, wherein the filtration unit is wetted prior to packaging.

10. The apparatus of claim 6, wherein the filtration unit comprises a one or more hollow fibers.

11. The apparatus of claim 10, wherein the one or more hollow fibers comprise a mixed cellulose ester, polysulfone, plastic polymers or some combination.

12. The apparatus of claim 10, wherein the one or more hollow fibers are open at the upper ends and closed at the lower ends or closed at the upper ends and open at the lower ends.

13. The apparatus of claim 6,
wherein the filtration unit is disposed with a first tube segment and the collection chamber is disposed within a second tube segment;
and wherein the first tube segment and the second tube segment are detachably connected to each other; or
wherein the filtration unit and the collection chamber are disposed within a single tube.

14. The apparatus of claim 6, wherein a source of the biological fluid comprises a source chamber upstream from the filtration unit and a needle to facilitate fluid communication with the filtration unit.

15. The apparatus of claim 14, further comprising a first vacuum port and a second vacuum port downstream from the filtration unit, wherein the first vacuum port and the second vacuum port are capable of coupling to a first vacuum collection chamber and a second vacuum collection chamber, respectively.

16. The apparatus of claim 14, wherein the source of the biological fluid further comprises a needle surrounded by a shroud.

17. The apparatus of claim 14, further comprising a first sample flow-through port in fluid communication with the filtration unit with a sample flow-through and a second sample flow-through in fluid communication with a sample permeate from the filtration unit.

18. The apparatus of claim 17, wherein the first downstream port and the second downstream port are complementary male and female connectors.

19. The apparatus of claim 17, wherein the first downstream port and the second downstream port are connected via a male taper-fitting and its mating female.

20. The apparatus of claim 6, wherein the filtration unit, the collection chamber, or both, further comprise one or more preservatives, chelating agents, stabilizers, EDTA, EGTA, thrombin, heparin, oxalate, fluoride, sodium citrate, acid-citrate-dextrose, sodium polyanethole sulfonate, anticoagulants, antibiotics, proteases or salts.

21. The apparatus of claim 6, wherein the interior surfaces of the filtration unit are positively charged or be negatively charged or the interior surfaces of the collection chamber are positively charged or negatively charged.

22. The apparatus of claim 6, wherein the apparatus does not require electrical power.

23. The apparatus of claim 6, wherein the collection chamber is a syringe.

24. The apparatus of claim 23, wherein the syringe comprises two separate concentric chambers and plungers.

25. The apparatus of claim 6, wherein the apparatus is adapted to couple in fluid connection to a needle within a shroud.

26. The apparatus of claim 6, wherein the biological fluid comprises whole blood, sputum, cerebrospinal fluid, tears, urine, feces, or a biopsy sample.

27. The apparatus of claim 6, wherein the selected biological material comprises cells, cellular organelles, bacteria, viruses, or cell-free DNA.

28. The apparatus of claim 6, wherein a source of the biological fluid is a vacuum blood collection tube.

29. The apparatus of claim 6, wherein the apparatus is configured to filter a sample of biological fluid with a volume of 4-10 ml.

30. The apparatus of claim 6, wherein the apparatus is disposable.

31. A filter kit comprising:
a filter apparatus comprising:
a filtration unit; and
a collection chamber in fluid communication with the filtration unit;
wherein a vacuum draws the biological fluid through the apparatus; and
wherein selected biological material comprised by the biological fluid is separated from a remainder of the biological fluid.

32. A method of filtering a biological fluid comprising:
connecting a filtration unit to a source of biological fluid including selected biological material;
connecting a collection chamber to the filtration unit;
drawing the biological fluid through the filtration unit with a vacuum; and
separating the selected biological material from a remainder of the biological fluid by filtration through the filtration unit.

33. The method of claim 32, wherein the selected biological material is substantially retained by the filtration unit and the remainder of the biological fluid is substantially passed to the collection chamber or the selected biological material is passed to the collection chamber and the remainder of the biological fluid is substantially retained by the filtration unit.

34. The method of claim 32, wherein the filtration unit comprises a hollow-fiber filtration unit, a membrane filtration unit, or a cassette filtration unit.

35. The method of claim 32, wherein the filtration unit comprises one or more hollow fibers.

36. The method of claim 32, wherein the one or more hollow fibers comprise a mixed cellulose ester, polysulfone, plastic polymers or some combination.
